(19) United States
(12) Patent Application Publication
(10) Pub. No.: US 2007/0298013 A1
(43) Pub. Date: Dec. 27, 2007

(54) Animal Nutritional Supplement and Method

(76) Inventor: Jay A. Altman, Fort Collins, CO (US)

Correspondence Address: A LAW FIRM, P.C., 8753 YATES DRIVE, SUITE 215, WESTMINSTER, CO, 80031, US

(21) Appl. No.: 11/767,345

(22) Filed: Jun. 22, 2007

Related U.S. Application Data
(60) Provisional application No. 60/805,705, filed on Jun. 23, 2006.

(51) Int. Cl. A61K 35/66; A61P 1/00

(52) U.S. Cl. 424/93.3; 424/93.44; 424/93.45; 424/93.46; 424/93.5; 424/93.51

(57) ABSTRACT

The disclosed device relates to an animal nutritional supplement and more particularly to a method of using an animal nutritional supplement to reduce the incidence and effects of one or more chronic or acute conditions associated with nutritional deficiencies, gastrointestinal sand accumulation, colitis, inflammatory bowel disease, bacterial colitis, gastrointestinal pH irregularities, colonic ulcers, colonic motility deficiencies, colic, gastrointestinal fermentation deficiencies, pathological changes of certain organs in equine, and possibly even physical aging. One embodiment of the animal nutritional supplement comprises psyllium in combination with a blend of synbiotics.

### CROSS REFERENCE APPLICATIONS

This application is a nonprovisional application claiming the benefits of provisional application No. 60/805,705 filed Jun. 23, 2006.

### FIELD OF ART

The disclosed device relates to an animal nutritional supplement and more particularly to a method of using an animal nutritional supplement to reduce the incidence and effects of chronic or acute conditions associated with one or more of, nutritional deficiencies, gastrointestinal sand accumulation, colitis, inflammatory bowel disease, bacterial colitis, gastrointestinal pH irregularities, colonic ulcers, colonic motility deficiencies, colic, gastrointestinal fermentation deficiencies, pathological changes of certain organs in equine, and possibly even physical aging. One embodiment of the disclosed animal nutritional supplement comprises psyllium in combination with a blend of synbiotics.

### BACKGROUND

Psyllium is the common name used for several members of the plant genus Plantago. Psyllium seeds are used commercially for the production of mucilage. Plantago seed mucilage is often referred to as husk or psyllium husk and is commonly used as a dietary fiber in humans because it is not digested in the small intestine. Psyllium is typically very rich in soluble fiber. Because psyllium mucilage absorbs excess water and can stimulate normal bowel elimination, it is commonly used as a laxative. Psyllium mucilage can also be used as a natural dietary fiber for animals. The dehusked seed that remains after the psyllium seed coat is milled away is known to be rich in starch and fatty acids. Not only is the seed an effective dietary fiber, it can be used as chicken feed and as cattle feed throughout the world.

Synbiotics refer to combination nutritional supplements comprising probiotics and prebiotics. Probiotics are defined as live microorganisms and yeasts that may beneficially affect a host upon ingestion by improving the balance of intestinal microflora. In general, probiotics produce organic acids such as lactic acid and acetic acid which inhibit the growth of pathogenic bacteria. Consequently, probiotics are believed to be useful in the treatment and prevention of conditions caused by pathogenic bacteria. Further, probiotics are believed to inhibit the growth and activity of putrefying bacteria and hence the production of toxic amine compounds. It is also believed that probiotic bacteria activate the immune function of the host.

Prebiotics are defined as nondigestible or selectively digestible food ingredients that may beneficially affect a host by selectively stimulating the growth and/or the activity of a limited number of bacteria in the digestive tract. Specifically, prebiotics selectively support the growth of beneficial bacterial species over pathogenic ones. Thus, to be effective, prebiotics should escape digestion in the upper gastrointestinal tract and be used by a limited number of the microorganisms comprising the colonic microflora. Typically, prebiotics do not directly colonize the digestive tract. Prebiotics are principally certain fibers, such as oligosaccharides, but can also comprise yeast, yeast cultures, fungal cultures and other feedstuffs.

In light of the beneficial effects of synbiotics, there has been interest in incorporating synbiotics into animal feeds, specifically equine feed. There are numerous equine supplements on the market containing synbiotics. Applicant has developed an animal supplement that comprises psyllium in combination with a blend of synbiotics. In another embodiment, psyllium and synbiotics may also be combined with smectite (e.g., hydrated sodium calcium aluminum silicate).

### SUMMARY OF THE DISCLOSURE

Before explaining the disclosed embodiments in detail, it is to be understood that the embodiments are not limited in application to the details of the particular arrangements shown, since other embodiments are possible. Also, the terminology used herein is for the purpose of description and not of limitation. The embodiments and features are described and illustrated in conjunction with systems, tools and methods which are meant to exemplify and to illustrate, not be limiting in scope.

The equine nutritional supplement can assist in sand clearance from the colon, can improve fermentation of feeds and forages, can increase and stabilize cecal/colonic pH, can decrease lactic acid build up in the cecum caused by high starch diets, can reduce colonic ulceration, can improve nutrient production and uptake, can increase phosphorous availability, and can stabilize the hind gut improving the chances of effective sand clearance. In certain formulas, the animal nutritional supplement may reduce detrimental bacterial toxins in the colon. Additionally, it appears that in some cases, the animal nutritional supplement may encourage hoof/foot growth.

An animal nutritional supplement is disclosed comprising psyllium in combination with a blend of synbiotics. In one embodiment, 95% (minimum purity) whole blond psyllium husks are used. However, other forms of psyllium may be used and still be within the scope of the disclosure. In addition, other ranges could be employed. For example, a study involving eight (8) equine indicated a use of 99.5% whole blond psyllium husks and synbiotics and provided success for sand clearance. The synbiotic microorganisms may be selected from one or more microorganisms based on their suitability toward animal consumption and their ability to improve the microbial balance in the animal intestine. The animal nutritional supplement has been used effectively in equines. The animal nutritional supplement may also be formulated with sweeteners and a flavored base to improve its taste. Fill and/or binding agents can aid in producing a form for administering the supplement to the target animal.

As stated above, synbiotics comprise both probotics and prebiotics. Examples of suitable synbiotic microorganisms can include yeasts such as Saccharomyces and their equivalents, molds such as Aspergillus and their equivalents, and bacteria such as the genera Bifidobacterium, Propionibacterium, Streptococcus, Enterococcus, Bacillus, Pediococcus, Lactobacillus and their equivalents. In some of the disclosed embodiments, synbiotic microorganisms and products such as Lactobacillus acidophilus, Lactobacillus casei, Lactobacillus salivarius, Lactobacillus plantarum, Lactobacillus Lactis, Lactobacillus rhamnosus, Lactobacillus rhamnosus GG, Lactobacillus pentosus WE7, Lactobacillus reuteri, Lactobacillus crispatus, Lactobacillus johnsonii, Lactobacillus equi, Bifidobacterium longum, Enterococcus faecium, Pediococcus acidilactici, Propionibacterium freudenreichii, Bacillus subtilis, Bacillus pumilus, Bacillus lichenformis, Streptococcus Cremoris, Streptococcus Diacetilactis, Aspergillus Oryzae, Ashbaya Gossypii, Propioni Bacterium, Trichoderma longibrachiatum, Saccharomyces cerevisiae, dried Saccharomyces cerevisiae fermentation solubles, Saccharomyces boulardii, dried Aspergillus niger fermentation extra, dried Trichoderma longibrachiatum fermentation extract, dried Bacillus subtillis fermentation extract, Active Dry Yeast, Brewers Dried Yeast, Glucomannoprotiens and Mannanoligosaccharides have been used. The synbiotic microorganisms are typically in dried granular form but any form can be utilized and still be within the scope of the disclosure.

Various amounts of synbiotics may be combined in a mixture; however supplied blends are available from distributors of these substances. For example, one embodiment comprises synbiotics from Alltech,® Inc. (i.e. product names are Bio-Mos,® Lacto-sacc® and Yea-Sacc® ¹⁰²⁶) Other sources could prove viable.

One embodiment of the present device comprises psyllium from about 78% to about 85% by weight and synbiotics from about 5% to about 8% by weight.

In one embodiment, the portion of synbiotics comprises dried Saccharomyces cerevisiae, dried Lactobacillus acidophilus, dried Enterococcus faecium, Brewers Dried Yeast, dried Saccharomyces cerevisiae, fermentation solubles, dried Aspergillus niger fermentation extra, dried Trichoderma longibrachiatum fermentation extract, dried Bacillus subtillis fermentation extract, fermentation solubles, glucomannoprotiens, mannanoligosaccharides. Additionally, the mixture can comprise binding agents, sweeteners and flavoring. The live cell additives comprise saccharomyces cerevisiae at a concentration of about 40 billion cells/lb, lactobacillus acidophlilis at a concentration of about 2.5 billion cells/lb and enterococcus faecium at a concentration of about 1.55 billion cells/lb.

Examples of suitable fill or binding agents can include lignin-based binding agents, molasses-based binding agents, calcium bentonite binding agents, gelatins, soy-based lecithin, casein, gluten, cellulose, wheat millrun and aqueous solutions. Suitable sweeteners can include natural sweeteners such as molasses and simple sugars such as sucrose and dextrose. Artificial sweeteners such as saccharin-based sweeteners, aspartame and sorbitol (glucitol) can also be employed. Equine have shown a tolerance for vanilla, licorice, cherry, citrus and apple flavoring. However, other flavoring can also be selected if desired. Secondary flavorings may be used as enhancing agents. For example, a combination of dry flavors and oil-based flavors may be used for extended aroma properties over the course of the product's recommended shelf-life.

Another embodiment of the present device may also include smectite. Smectite may either be dioctahedral smectite, trioctahedral smectite or a combination of both which is commonly known as DTO Smectite (Di, Tri Octahedral). Smectite may aid in reducing detrimental bacterial toxins in the colon. This embodiment of the present device comprises psyllium from about 25% to about 85% by weight, smectite from about 10% to about 70% by weight and synbiotics from about 2% to about 10% by weight. An alternate embodiment comprises psyllium from about 4% to about 85% by weight, smectite from about 10% to about 70% by weight and synbiotics from about 25% to about 30% by weight

It is to be understood that the presently disclosed animal nutritional supplements are not limited to the specific compounds, concentrations and/or ranges noted above. For example, other embodiments comprise synbiotics ranging in concentration greater than that of psyllium and/or smectite, and smectite could range in concentration greater than that of psyllium and/or synbiotics.

The animal nutritional supplements may be administered in various forms. For example, equine may tolerate pellets, crumbles, flakes, powder, granules, tablets, liquid, paste or solution. In addition, the supplement may be administered individually, as a feed additive, syringed by mouth (per os) or pasted by mouth (per os). It has been shown that the animal nutritional supplement is effective in a pelleted form which can be mixed into food, such as grain.

The quantity of the formulation administered to an animal can be dependent on various factors such as general health, weight, age, state of nutrition, and/or type and severity of the ailment. For example, in equine ranging from 900 to 1400 pounds, a single standard dosage of the formulation described above may be between about four ounces to about eight ounces daily. An equine having a severe ailment, however, could require two or more standard dosages. In some cases, administering the nutritional supplement may only be necessary for a short time period of time but in many cases the physical condition of the horse and the environmental and management conditions surrounding the horse may require either continuous or intermittent dosing for treatment even after symptoms disappear.

To produce the animal nutritional supplement, conventional means may be employed. One method comprises 1) mixing the dry ingredients until a mixture having a uniform consistency is achieved; 2) adding wet ingredients and/or moisture to the first mixture in order to reach an overall moisture content suitable for the pelletizing process; and 3) pelletizing the wet/dry mixture.

Mixing should continue until uniform moisture throughout the product is observed. The product typically remains at room temperature throughout the initial mixing process. It may be desirable to take a sample of the first mixture to determine the moisture content due to environmental conditions at the time of processing. In some cases, spot samples are taken regularly to confirm that the overall moisture content does not drop. If the moisture content falls below the desired level, the moisture content can be restored to optimum levels by adding additional moisture to the remaining mixed product. During the pelletizing process, the temperature of the mixture/finished pellets can reach a range of about 90° F. (about 32.2° C.) to about 105° F. (about 40.6° C.). The finished pellets are typically transferred to a cooling bin, where pellets are cooled by a forced airflow or other means. Once cooled to room temperature, the pellets are placed into holding bins and prepared for final packaging.

While a number of exemplifying features and embodiments have been discussed above, those of skill in the art will recognize certain modifications, permutations, additions and subcombinations thereof. Other alternative embodiments of the present device and method could be easily employed by those skilled in the art. No limitation with respect to the specific embodiments disclosed herein is intended or should be inferred. Each device embodiment described herein has numerous equivalents.

I claim:

1. An animal nutritional supplement comprising:
psyllium;
a synbiotic; and
wherein said synbiotic comprises one or more of Lactobacillus acidophilus, Lactobacillus casei, Lactobacillus salivarius, Lactobacillus plantarum, Lactobacillus Lactis, Lactobacillus rhamnosus, Lactobacillus rhamnosus GG, Lactobacillus pentosus WE7, Lactobacillus reuteri, Lactobacillus crispatus, Lactobacillus johnsonii, Lactobacillus equi, Bifidobacterium longum, Enterococcus faecium, Pediococcus acidilactici, Propionibacterium freudenreichii, Bacillus subtilis, Bacillus pumilus, Bacillus lichenformis, Streptococcus Cremoris, Streptococcus Diacetilactis, Aspergillus Oryzae, Ashbaya Gossypii, Propioni Bacterium, Trichoderma longibrachiatum, Saccharomyces cerevisiae, dried Saccharomyces cerevisiae fermentation solubles, Saccharomyces boulardii, dried Aspergillus niger fermentation extra, dried Trichoderma longibrachiatum fermentation extract, dried Bacillus subtillis fermentation extract, Active Dry Yeast, Brewers Dried Yeast, Glucomannoprotiens and Mannanoligosaccharides.

2. The animal nutritional supplement of claim 1, wherein said psyllium comprises about 95% whole blond psyllium husks.

3. The animal nutritional supplement of claim 1 further comprising smectite.

4. The animal nutritional supplement of claim 3, wherein said smectite is selected from the group consisting of dioctahedral smectite, trioctahedral smectite, di, tri octahedral smectite, and mixtures thereof.

5. The animal nutritional supplement of claim 1 further comprising a fill or binding agent.

6. The animal nutritional supplement of claim 5, wherein said fill or binding agent comprises one or more of lignin-based binding agents, molasses-based binding agents, calcium bentonite binding agents, gelatins, soy-based lecithin, casein, gluten, cellulose, wheat millrun, and aqueous solutions.

7. The animal nutritional supplement of claim 5 further comprising a sweetener.

8. The animal nutritional supplement of claim 7, wherein said sweetener is selected from the group consisting of molasses, sucrose, dextrose, aspartame, sorbitol, saccharine and mixtures thereof.

9. The animal nutritional supplement of claim 7 further comprising flavoring.

10. The animal nutritional supplement of claim 9, wherein said flavoring is selected from the group consisting of vanilla, licorice, cherry, apple, citrus, and mixtures thereof.

11. The animal nutritional supplement of claim 9 further comprising a secondary flavoring to extend an aromatic property.

12. The animal nutritional supplement of claim 3 further comprising a fill or binding agent, a sweetener and flavoring.

13. The animal nutritional supplement of claim 1, wherein said animal nutritional supplement is pelleted.

14. A pelletized animal nutritional supplement comprising:
about 78% to about 85% by weight of psyllium; and
about 5% to about 8% by weight of synbiotic, and
wherein said synbiotic comprises one or more of dried Saccharomyces cerevisiae, dried Lactobacillus acidophilus, dried Enterococcus faecium, Brewers Dried Yeast, dried Saccharomyces cerevisiae, fermentation solubles, dried Aspergillus niger fermentation extra, dried Trichoderma longibrachiatum fermentation extract, dried Bacillus subtillis fermentation extract, fermentation solubles, glucomannoprotiens, and mannanoligosaccharides.

15. The animal nutritional supplement of claim 14, wherein said psyllium comprises about 95% whole blond psyllium husks.

16. The animal nutritional supplement of claim 14 further comprising smectite.

17. The animal nutritional supplement of claim 14 further comprising a fill or binding agent.

18. The animal nutritional supplement of claim 17 further comprising a sweetener.

19. The animal nutritional supplement of claim 18 further comprising flavoring.

20. The animal nutritional supplement of claim 16 further comprising a fill or binding agent, a sweetener and flavoring.

21. An animal nutritional supplement comprising:
about 4% to about 85% by weight of psyllium;
about 25% to about 30% by weight of synbiotics;
about 10% to about 70% by weight of smectite; and
wherein said synbiotic comprises one of more of dried Saccharomyces cerevisiae, dried Lactobacillus acidophilus, dried Enterococcus faecium, Brewers Dried Yeast, dried Saccharomyces cerevisiae, fermentation solubles, dried Aspergillus niger fermentation extra, dried Trichoderma longibrachiatum fermentation extract, dried Bacillus subtillis fermentation extract, fermentation solubles, glucomannoprotiens, and mannanoligosaccharides.

22. The animal nutritional supplement of claim 21, wherein said psyllium comprises about 95% whole blond psyllium husks.

23. The animal nutritional supplement of claim 21, wherein said smectite is selected from the group consisting of dioctahedral smectite, trioctahedral smectite, di, tri octahedral smectite, and mixtures thereof.

24. The animal nutritional supplement of claim 21 further comprising a fill or binding agent.

25. The animal nutritional supplement of claim 24 further comprising a sweetener.

26. The animal nutritional supplement of claim 25 further comprising flavoring.

27. A method for treating a digestive system of an equine comprising the step of administering an animal nutritional supplement to an equine, said supplement comprising a combination of psyllium and synbiotics, wherein said synbiotics comprise one or more of Lactobacillus acidophilus, Lactobacillus casei, Lactobacillus salivarius, Lactobacillus plantarum, Lactobacillus Lactis, Lactobacillus rhamnosus, Lactobacillus rhamnosus GG, Lactobacillus pentosus WE7, Lactobacillus reuteri, Lactobacillus crispatus, Lactobacillus johnsonii, Lactobacillus equi, Bifidobacterium longum, Enterococcus faecium, Pediococcus acidilactici, Propionibacterium freudenreichii, Bacillus subtilis, Bacillus pumilus, Bacillus lichenformis, Streptococcus Cremoris, Streptococcus Diacetilactis, Aspergillus Oryzae, Ashbaya Gossypii, Propioni Bacterium, Trichoderma longibrachiatum, Saccharomyces cerevisiae, dried Saccharomyces cerevisiae fermentation solubles, Saccharomyces boulardii, dried Aspergillus niger fermentation extra, dried Trichoderma longibrachiatum fermentation extract, dried Bacillus subtillis fermentation extract, Active Dry Yeast, Brewers Dried Yeast, Glucomannoprotiens and Mannanoligosaccharides.
